(19) United States
(12) Patent Application Publication
(10) Pub. No.: US 2021/0279554 A1
(43) Pub. Date: Sep. 9, 2021

(54) METHOD AND APPARATUS FOR MONITORING PHYSICAL ACTIVITY

(71) Applicant: SAMSUNG ELECTRONICS CO., LTD., Suwon-si (KR)

(72) Inventor: Nabil Ibtehaz, Dhaka (BD)

(73) Assignee: SAMSUNG ELECTRONICS CO., LTD., Suwon-si (KR)

(21) Appl. No.: 17/187,230

(22) Filed: Feb. 26, 2021

(30) Foreign Application Priority Data
Mar. 3, 2020 (KR) 10-2020-0026793

(51) Int. Cl. G06N 3/04; G06N 3/08; G16H 40/67; A61B 5/11; A61B 5/00

(52) U.S. Cl. CPC G06N 3/0454; G06N 3/08; G16H 40/67; A61B 5/1118; H04W 88/02; A61B 5/7267; A61B 5/6898; A61B 5/6817; A61B 5/7278; A61B 5/1123

(57) ABSTRACT

An apparatus for monitoring a physical activity includes: a memory storing one or more instructions; and at least one processor configured to execute the one or more instructions stored in the memory to: obtain first type sensor data from a first type wearable sensor; obtain second type sensor data from a second type wearable sensor; and identify the physical activity of a user by using at least one artificial intelligence learning model, the first type sensor data, and the second type sensor data.

### CROSS-REFERENCE TO RELATED APPLICATION

This application is based on and claims priority under 35 U.S.C. § 119 to Korean Patent Application No. 10-2020-0026793, filed on Mar. 3, 2020, in the Korean Intellectual Property Office, the disclosure of which is incorporated by reference herein in its entirety.

### BACKGROUND

### 1. Field

The disclosure relates to a method and an apparatus for monitoring a physical activity.

### 2. Description of Related Art

Recently, wearable devices, such as smart watches, smart bands, fitness trackers, etc., have been widely distributed, and technologies for monitoring user's physical activities by using an acceleration sensor, a gyro sensor, etc. included in the wearable devices have been developed. Along with this, many insurance companies, finance firms, corporates, schools, etc. have provided compensations based on data related to physical activities offered by the wearable devices, and the physical activities-related data provided by the wearable devices may also be used as the evidence in court. However, the monitoring of the physical activities via the wearable devices may be inaccurate. Also, research has been conducted to provide a method of spoofing fitness data that allows a user to create fake fitness data to qualify for insurance discounts. Therefore, techniques for relatively more accurately monitoring a user's physical activities by using wearable devices and preventing data spoofing are required.

An artificial intelligence (AI) system is a system configured to perform self-learning and self-determination and get smarter, unlike a previous rule-based smart system. The more the AI system is used, the higher the recognition rate of the AI system, and the AI system may more accurately understand the user's taste. Thus, the previous rule-based smart system has been gradually replaced by a deep learning-based AI system. AI technologies are composed of machine learning and element technologies using the machine learning. Machine learning is an algorithm technology that classifies or learns characteristics of input data on its own. The element technology uses machine learning algorithms, such as deep learning, etc., and includes technical fields of linguistic understanding, visual comprehension, inference or prediction, knowledge representation, operation control, etc.

### SUMMARY

Provided are a method and an apparatus for monitoring a physical activity by using a wearable sensor, in order to relatively more accurately monitor user's physical activities.

According to an embodiment of the disclosure, there is provided an apparatus for monitoring a physical activity, including: a memory storing one or more instructions; and at least one processor configured to execute the one or more instructions stored in the memory, to: obtain first type sensor data from a first type wearable sensor; obtain second type sensor data from a second type wearable sensor; and identify the physical activity of a user by using at least one artificial intelligence learning model, the first type sensor data, and the second type sensor data.

The at least one artificial intelligence learning model may include a first artificial intelligence learning model and a second artificial intelligence learning model, and the at least one processor may be further configured to execute the one or more instructions to: identify the physical activity of the user by inputting the first type sensor data into the first artificial intelligence learning model; and verify the identified physical activity by inputting the second type sensor data into the second artificial intelligence learning model.

The at least one processor may be further configured to execute the one or more instructions to verify the identified physical activity by inputting the second type sensor data into the second artificial intelligence learning model, when the physical activity of the user identified based on the first type sensor data is one of a plurality of pre-defined physical activities.

The at least one artificial intelligence learning model may include a first artificial intelligence learning model and a second artificial intelligence learning model, and the at least one processor may be further configured to execute the one or more instructions to: identify a first physical activity of the user by inputting the first type sensor data into the first artificial intelligence learning model; identify a second physical activity of the user by inputting the second type sensor data into the second artificial intelligence learning model; and determine whether or not the first physical activity corresponds to the second physical activity correspond.

The at least one artificial intelligence learning model may include a first artificial intelligence learning model, and the at least one processor may be further configured to execute the one or more instructions to identify the physical activity of the user by inputting the first type sensor data and the second type sensor data into the first artificial intelligence learning model.

When a current value of the second type sensor data is not obtained, the at least one processor is further configured to execute the one or more instructions to identify the physical activity of the user by inputting, into the at least one artificial intelligence learning model, an estimated value of the second type sensor data, the estimated value being estimated based on at least one of a previous value of the second type sensor data or the first type sensor data.

The first type wearable sensor may include a motion sensor, and the first type sensor data may include motion sensor data obtained from the motion sensor, and the second type wearable sensor may include a biomedical sensor, and the second type sensor data may include biomedical sensor data obtained from the biomedical sensor.

The first type wearable sensor may include a first motion sensor worn on a first body part of the user, and the first type sensor data includes first body part motion sensor data obtained from the first motion sensor worn on the first body part of the user, and the second type wearable sensor may include a second motion sensor worn on a second body part of the user, and the second type sensor data may include second body part motion sensor data obtained from the second motion sensor worn on the second body part of the user.

The at least one artificial intelligence learning model may include a first artificial intelligence learning model, and the at least one processor may be further configured to execute the one or more instructions to identify a whole body physical activity of the user by inputting the first body part motion sensor data and the second body part motion sensor data into the first artificial intelligence learning model.

The first type wearable sensor may include a smartphone motion sensor included in a smartphone, and the first type sensor data may include smartphone motion sensor data obtained from the smartphone motion sensor, the at least one artificial intelligence learning model may include a first artificial intelligence learning model, and the at least one processor may be further configured to execute the one or more instructions to identify a body part, on which the smartphone is worn, based on the smartphone motion sensor data, by using the first artificial intelligence learning model.

The first body part and the second body part may not include a torso, the at least one artificial intelligence learning model may include a first artificial intelligence learning model, and the at least one processor may be further configured to execute the one or more instructions to identify a motion of the torso of the user based on the first body part motion sensor data and the second body part motion sensor data, by using the first artificial intelligence learning model.

The first type wearable sensor may include a first side motion sensor of the first body part at a first side of the first body part, and the first type sensor data may include first side motion sensor data of the first body part obtained from the first side motion sensor of the first body part. The second type wearable sensor may include a second side motion sensor of the second body part at a second side of the second body part, and the second type sensor data may include second side motion sensor data of the second body part obtained from the second side motion sensor of the second body part, wherein the second side is opposite to the first side. The at least one processor may be further configured to execute the one or more instructions to identify the motion of the torso of the user based on the first side motion sensor data of the first body part and the second side motion sensor data of the second body part, by using the first artificial intelligence learning model.

The second type wearable sensor may include an earphone motion sensor included in an earphone, and the second type sensor data may include earphone motion sensor data obtained from the earphone motion sensor.

The earphone motion sensor may include a left earphone motion sensor included in a left earphone portion and a right earphone motion sensor included in a right earphone portion, and the earphone motion sensor data may include left earphone motion sensor data obtained from the left earphone motion sensor and right earphone motion sensor data obtained from the right earphone motion sensor.

The first type wearable sensor may include a first side motion sensor at a first side of the first body part, and the first type sensor data includes first side motion sensor data obtained from the first side motion sensor, the at least one artificial intelligence learning model may include a first artificial intelligence learning model, and the at least one processor may be further configured to execute the one or more instructions to identify a motion of a second side of the first body part based on the first side motion sensor data and the earphone motion sensor data, by using the first artificial intelligence learning model, wherein the second side may be opposite to the first side.

The least one processor may be further configured to execute the one or more instructions to determine vertical symmetry of the physical activity of the user based on the earphone motion sensor data.

The first type wearable sensor may include a one-sided motion sensor at a side of the body part, and the first type sensor data includes one-sided motion sensor data obtained from the one-sided motion sensor, the at least one artificial intelligence learning model may include a first artificial intelligence learning model, and the at least one processor may be further configured to execute the one or more instructions to: identify the physical activity of the user by inputting the one-sided motion sensor data into the first artificial intelligence learning model; and verify the identified physical activity of the user based on the determined vertical symmetry.

The first type wearable sensor may include a one-sided motion sensor at a side of the body part, and the first type sensor data may include one-sided motion sensor data obtained from the one-sided motion sensor, the at least one artificial intelligence learning model may include a first artificial intelligence learning model, and the at least one processor may be further configured to execute the one or more instructions to identify the physical activity of the user by inputting the determined vertical symmetry and the one-sided motion sensor data into the first artificial intelligence learning model.

According to an embodiment of the disclosure, there is provided an operating method of an apparatus for monitoring a physical activity, including: obtaining first type sensor data from a first type wearable sensor; obtaining second type sensor data from a second type wearable sensor; and identifying the physical activity of a user by using at least one artificial intelligence learning model, the first type sensor data, and the second type sensor data.

The at least one artificial intelligence learning model may include a first artificial intelligence learning model and a second artificial intelligence learning model, and the operating method may further comprises: identifying the physical activity of the user by inputting the first type sensor data into the first artificial intelligence learning model; and verifying the identified physical activity by inputting the second type sensor data into the second artificial intelligence learning model, when the physical activity of the user identified based on the first type sensor data is one of a plurality of pre-defined physical activities.

According to an embodiment of the disclosure, there is provided a non-transitory computer-readable recording medium having recorded thereon a computer program that is executable by at least one processor to perform the operating method.

According to an embodiment of the disclosure, there is provided a fitness tracking device, including: a memory storing one or more instructions; a motion sensor configured to obtain a motion sensor signal by detecting a movement of a user of the fitness tracking device; a biomedical sensor configured to obtain a biomedical signal from the user; and at least one processor configured to execute the one or more instructions stored in the memory, to: identify a physical activity of the user by inputting the motion sensor signal to a first artificial intelligence learning model; determine whether the physical activity corresponds to one of a plurality of predefined physical activities; and based on the physical activity corresponding to the one of the plurality of predefined physical activities, verify accuracy of an identification of the physical activity that is identified by the first artificial intelligence learning model, by inputting the biomedical signal to a second artificial intelligence learning model.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic block diagram of a structure of an apparatus for monitoring a physical activity, according to an embodiment of the disclosure;

FIGS. 2 through 4 are respectively schematic flowcharts of an operating method of an apparatus for monitoring a physical activity, according to an embodiment of the disclosure;

FIG. 5 is a schematic view of a method, performed by an apparatus for monitoring a physical activity, according to an embodiment of the disclosure, of determining a physical activity of a user based on motion sensor data;

FIG. 6 is a schematic view of a method, performed by an apparatus for monitoring a physical activity, according to an embodiment of the disclosure, of verifying, based on biomedical sensor data, a physical activity of a user determined based on motion sensor data;

FIG. 7 is a schematic view of a method of training an artificial intelligence (AI) learning model by using motion sensor data and biomedical sensor data as training data, according to an embodiment of the disclosure;

FIG. 8 is a schematic view of a method, performed by an apparatus for monitoring a physical activity, according to an embodiment of the disclosure, of determining a physical activity of a user based on motion sensor data and biomedical sensor data;

FIG. 9 is a view of electrocardiogram (ECG) signals when a user takes a rest and when the user does exercise;

FIG. 10 is a view of statistical characteristics of ECG signals when a user takes a rest and when the user does exercise;

FIG. 11 is a view of frequency spectrums of ECG signals when three users take a rest and when the users do exercise;

FIG. 12 is a view of a confusion matrix showing performance of an apparatus for monitoring a physical activity using motion sensor data and biomedical sensor data together; and

FIG. 13 is a schematic view of a method, performed by an apparatus for monitoring a physical activity, according to an embodiment of the disclosure, of determining a physical activity of a user based on data of sensors worn on a plurality of body parts.

### DETAILED DESCRIPTION

Embodiments of the disclosure will be described in detail with reference to the accompanying drawings in order to clearly describe the technical concept of the disclosure. When describing the disclosure, well-known functions or components in the art will not be described in detail, when it is determined that the detail descriptions thereof may unnecessarily blur the concept of the disclosure. In the drawings, components having substantially the same functional configurations are given like reference numerals and like signs as possible, even when the components are illustrated in different drawings. When it is necessary for convenience of explanation, an apparatus and a method will be described together. Operations of the disclosure do not necessarily have to be performed in described orders and may be performed in a parallel fashion, a selective fashion, or a separate fashion.

Throughout the disclosure, the expression “at least one of a, b or c” indicates only a, only b, only c, both a and b, both a and c, both b and c, all of a, b, and c, or variations thereof.

While such terms as “first,” “second,” etc., may be used to describe various elements, such elements must not be limited to the above terms. The above terms may be used only to distinguish one element from another.

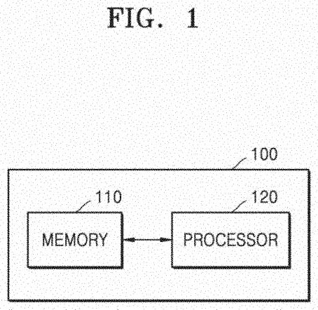

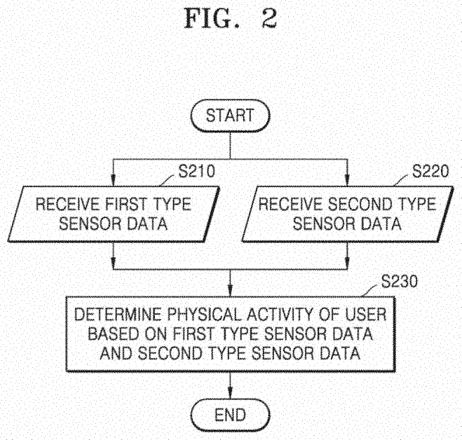

FIG. 1 is a schematic block diagram of a structure of an apparatus 100 for monitoring a physical activity, according to an embodiment of the disclosure. Referring to FIG. 1, the apparatus 100 for monitoring the physical activity, according to an embodiment of the disclosure, may include a memory 110 storing one or more instructions and a processor 120 configured to execute the one or more instructions stored in the memory 110. The memory 110 may include a single memory or a plurality of memories. The processor 120 may include a single processor or a plurality of processors. An operation of the apparatus 100 for monitoring the physical activity, the operation being performed by the processor 120, is described in detail hereinafter with reference to FIG. 2, etc.

FIG. 2 is a schematic flowchart of an operating method of the apparatus 100 for monitoring the physical activity, according to an embodiment of the disclosure. Referring to FIG. 2, the apparatus 100 for monitoring the physical activity may receive first type sensor data from a first type wearable sensor in operation S210 and may receive second type sensor data from a second type wearable sensor in operation S220.

Here, the wearable sensors refer to sensors worn on the body of a user. Wearing a sensor on the body refers to having the sensor directly contact a particular body part or having the sensor fastened close thereto. Wearing a sensor on the body may indicate wearing the sensor on the body or wearing a device including the sensor on the body. The device including the sensor may include not only general wearable devices, such as a smart watch, but also any article which may be fastened to or placed on the body of a user, or may be carried by the user. For example, a sensor may be included in a bracelet, a necklace, an earring, a ring, a watch, glasses, sunglasses, a head-mounted display (HMD), a hat, a helmet, a hair band, a joint protector, gloves, shoes, a belt, or clothes, etc. Wearing a sensor on the body may not only indicate having the sensor (or a device including the sensor) fastened to a particular part of the body such that the sensor is not movable, but may also indicate having the sensor disposed such that a location of the sensor is not greatly deviated from a particular part of the body, as when the sensor is put in a clothing pocket. For example, when a smartphone is kept in a trouser pocket, various sensors included in the smartphone may be regarded as wearable sensors worn on legs.

A first type wearable sensor and a second type wearable sensor may indicate different types of wearable sensors. The different types of wearable sensors may include sensors sensing different types of physical quantities, sensors included in different types of apparatuses, sensors worn on different body parts, or the like. First type sensor data and second type sensor data may indicate different types of sensor data. The different types of sensor data may include data indicating different types of physical quantities, data sensed by different types of apparatuses, data sensed from different body parts, or the like.

The apparatus 100 for monitoring the physical activity may determine a physical activity of a user by inputting the first type sensor data and the second type sensor data into at least one artificial intelligence (AI) learning model, in operation S230. The AI learning model may determine the physical activity of the user based on the first type sensor data and the second type sensor data. In this case, the physical activity of the user may be determined by using the different types of sensor data, and thus, the physical activity may be more accurately determined than a case in which one type of sensor data is used. The physical activity may include, for example, taking exercise, taking a rest, running, performing a treadmill exercise, riding a bike, playing ski, boarding a vehicle, performing a push-up exercise, performing a bench-press exercise, performing a squat exercise, performing a kettlebell swing exercise, performing a dumbbell curl exercise, performing a buffy test exercise, jump-roping, performing an aerobic exercise, performing an anaerobic exercise, playing football, playing basketball, swimming, playing taekwondo, playing yoga, dancing, sleeping, dining, working, and the like. The AI learning model may be executed by the processor 120.

The AI learning model may be formed by taking into account an application field of the AI learning model, the purpose of learning, or the computer performance of an apparatus. The AI learning model may be a learning model that is trained by using, as an AI algorithm, at least one of machine learning, neural networks, genes, deep-learning, or a classification algorithm. For example, at least one of a convolutional neural network (CNN) model, a deep neural network (DNN) model, a recurrent neural network (RNN) model, a restricted Boltzmann machine (RBM) model, a deep belief network (DBN) model, a bidirectional recurrent deep neural network (BRDNN) model, or a deep Q-network model may be used as the AI learning model.

The AI learning model has the characteristics that the AI learning model is formed via learning. That the AI learning model is formed via learning denotes that an AI model configured to perform a desired function (or purpose) is formed by training a basic AI model by using a plurality of pieces of training data based on a learning algorithm. The training operation may be directly performed by the apparatus 100 for monitoring the physical activity or by an additional server and/or an additional system. Examples of the learning algorithm may include supervised learning, unsupervised learning, semi-supervised learning, or reinforcement learning.

The AI learning model may include a plurality of neural network layers. The plurality of neural network layers may include a plurality of nodes that respectively have a plurality of weight values, and may perform calculation using a calculation result of a previous layer and the plurality of weight values. The plurality of weight values of the plurality of neural network layers may be optimized based on learning results of the AI learning model. For example, the plurality of weight values may be modified and refined to reduce or minimize the loss value or the cost value obtained by the AI learning model during a learning process.

The AI learning model may correspond to a model that is trained to determine a physical activity based on sensor data as training data, the sensor data including the first type sensor data and the second type sensor data which are collected from the first type wearable sensor and the second type wearable sensor during various physical activities of a person wearing the first and second types wearable sensors. The AI learning model may include a first AI learning model and a second AI learning model. The first AI learning model may be trained by using, as the training data, the first type sensor data, which is collected from the first type wearable sensor when a person wearing the first type wearable sensor performs various physical activities, and the second AI learning model may be trained by using, as the training data, the second type sensor data, which is collected from the second type wearable sensor when a person wearing the second type wearable sensor performs various physical activities.

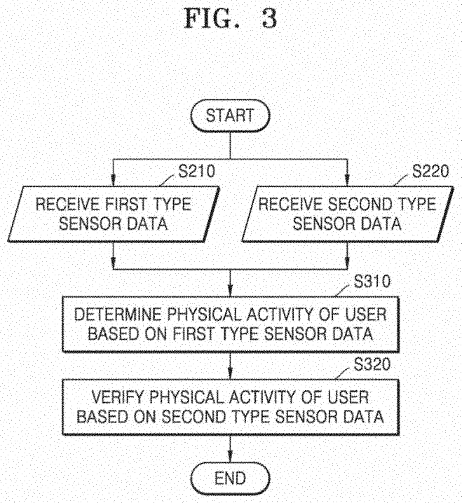

FIG. 3 is a schematic flowchart of an operating method of the apparatus 100 for monitoring the physical activity, according to an embodiment of the disclosure. Referring to FIG. 3, the AI learning model may determine a physical activity of a user by inputting the first type sensor data into the first AI learning model in operation S310 and may verify the physical activity, which is determined based on the first type sensor data, by inputting the second type sensor data into the second AI learning model, in operation S320.

According to an embodiment of the disclosure, when the AI learning model verifies the physical activity determined based on the first type sensor data, the AI learning model may verify the physical activity determined based on the first type sensor data, by inputting both of the first type sensor data and the second type sensor data into the second AI learning model.

According to another embodiment, when the AI learning model verifies the physical activity determined based on the first type sensor data, the AI learning model may compare a physical activity determined by inputting the second type sensor data into the second AI learning model with the physical activity determined based on the first type sensor data. In other words, the AI learning model may determine a first physical activity of the user by inputting the first type sensor data into the first AI learning model, may determine a second physical activity of the user by inputting the second type sensor data into the second AI learning model, and may determine whether or not the first physical activity and the second physical activity correspond to each other. The determining of the first physical activity and the determining of the second physical activity do not have to be performed in the stated order.

Here, the expressions of the first physical activity and the second physical activity do not indicate that the physical activities are different types of physical activities. Rather, the expressions indicate that the physical activities are determined based on different types of sensor data. The first physical activity and the second physical activity may be the same physical activity. That statement that the first physical activity and the second physical activity correspond to each other may denote that both of the first and the second physical activities are the same, and may also denote that one physical activity is included in the other physical activity, or both of the first and the second physical activities belong to a same category group. For example, when the first physical activity determined based on the first type sensor data is “a running exercise,” and the second physical activity determined based on the second type sensor data is “an aerobic exercise,” it may be determined that the first physical activity is rightly determined. When the second physical activity is “sleeping,” when the first physical activity determined based on the first type sensor data is “running,” it may be determined that the first physical activity is wrongly determined. The second physical activity determined based on the second type sensor data may simply correspond to either an exercise or a non-exercise. The memory 110 may store a list of a plurality of different types of physical activities, and corresponding category groups are assigned to each of the plurality of different types of physical activities. For example, the memory 110 may store information indicating that the “running exercise” and the “aerobic exercise” belong to activity category group 1, and the “sleeping” may belong to activity category group 2.

The AI learning model may input the physical activity determined based on the first type sensor data into the second AI learning model. That is, the AI learning model may input the physical activity determined based on the first type sensor data and the second type sensor data into the second AI learning model, in order to verify the physical activity determined based on the first type sensor data. Here, the second AI learning model may correspond to a model which is trained by using, as training data, the second type sensor data collected from the second type wearable sensor when a person wearing the second type wearable sensor performs various physical activities, and a type of a corresponding physical activity. An output of the second AI learning model may be related to whether a physical activity is true or false or may be related to a finally determined physical activity. According to the physical activity determined based on the first type sensor data, the second AI learning model may be selected from among a plurality of AI learning models. Each AI learning model may correspond to a model that is trained by using, as training data, the second type sensor data collected when the user performs a corresponding physical activity and when the user does not perform the corresponding physical activity. Thus, a threshold value for distinguishing a case in which a specific physical activity is performed and a case in which the specific physical activity is not performed distinguished may be adaptively determined.

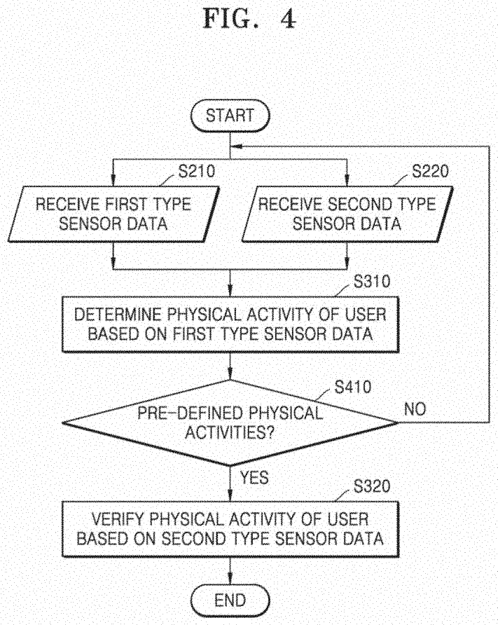

FIG. 4 is a schematic flowchart of an operating method of the apparatus 100 for monitoring the physical activity, according to an embodiment of the disclosure. Referring to FIG. 4, the AI learning model may verify the determined physical activity, only when the physical activity of the user, which is determined based on the first type sensor data, is included in pre-defined physical activities. That is, the AI learning model may determine the physical activity of the user by inputting the first type sensor data into the first AI learning model in operation S310, may determine whether or not the determined physical activity is included in the pre-defined physical activities in operation S410, and, when the determined physical activity is included in the pre-defined physical activities, may input the second type sensor data into the second AI learning model to verify the determined physical activity in operation S320. In this case, the amount of calculations and the power consumption may be reduced, because, in normal occasions when the user does not engage in any of the pre-defined physical activities, operation S320 may be omitted and only the processing based on the first type sensor data and the first AI learning model may be performed, and operation S320 may be performed only when it is determined that the user performs a specific physical activity based on the first type sensor data and the first AI learning model.

For example, only when the physical activity of the user, which is determined based on the first type sensor data, is included in exercises, the AI learning model may determine, based on the second type sensor data, whether or not the physical activity of the user is rightly determined. As another example, only when the physical activity of the user, which is determined based on the first type sensor data, is included in anaerobic exercises, the AI learning model may determine, based on the second type sensor data, whether or not the physical activity of the user is rightly determined.

The AI learning model may determine the physical activity of the user by inputting the first type sensor data and the second type sensor data together into one AI learning model. That is, the AI learning model may include the first AI learning model and may determine the physical activity of the user by inputting the first type sensor data and the second type sensor data into the first AI learning model. Here, the first AI learning model may correspond to a model which is trained by using, as training data, the first type sensor data and the second type sensor data collected from the first type wearable sensor and the second type wearable sensor when a person wearing the first and second types wearable sensors performs various physical activities.

The descriptions have been given above based on the case in which there are two types of wearable sensors. However, there may be three or more than three types of wearable sensors. For example, the apparatus 100 for monitoring the physical activity may receive the first type sensor data from the first type wearable sensor, the second type sensor data from the second type wearable sensor, and third type sensor data from a third type wearable sensor. The AI learning model may determine physical activities respectively indicated by the three types of sensor data by using three AI learning models respectively corresponding to the three types of sensor data and may determine whether or not the determined physical activities correspond to one another. The AI learning model may be trained to process two or more types of sensor data from among the three types of sensor data. The AI learning model may determine the physical activity based on at least one of the three types of sensor data and verify the determined physical activity based on the other types of sensor data. Hereinafter, for convenience of explanation, an example in which two types of sensors are used will be mainly described. However, the descriptions below may also be applied to an example in which three or more than three types of sensors are used.

As described above, the first type wearable sensor and the second type wearable sensor may correspond to sensors configured to sense different types of physical quantities. For example, the first type wearable sensor may include a motion sensor, and the second type wearable sensor may include a biomedical sensor. The motion sensor may be configured to sense a motion and may include an acceleration sensor, a gyro sensor, a geomagnetic sensor, or an atmospheric pressure sensor. When the motion sensor is worn on a specific body part of a user, the motion sensor may sense a motion of the corresponding body part. The biomedical sensor may be configured to sense a biometric signal and may include an electrocardiogram (ECG) sensor, a photoplethysmogram (PPG) sensor, an electroencephalogram (EEG) sensor, an electromyogram (EMG) sensor, or an electrooculogram (EOG) sensor.

The apparatus 100 for monitoring the physical activity may receive motion sensor data from the motion sensor worn on the user, may receive biomedical sensor data from the biomedical sensor worn on the user, and may input the motion sensor data and the biomedical sensor data to the AI learning model portion, to determine the physical activity of the user. In particular, the physical activity of the user may be determined by using the motion sensor data and the biomedical sensor data together, and thus, compared to when only the motion sensor data is used or when only the biomedical sensor data is used, the physical activity may be more accurately determined. In particular, ECG signals and PPG signals are greatly affected by a physical activity of a human being. Thus, when using the ECG sensor or the PPG sensor, the physical activity may be relatively more accurately determined.

For example, when the physical activity is determined based on only the motion sensor data, it may be determined that a user does exercise in all of three cases in which a user performs an arm workout by holding a dumbbell, in which a user repeatedly performs an arm workout with empty hand, and in which a smart watch is fastened to a metronome and is allowed to move. However, when the biomedical sensor data is used, the three cases may be distinguished from one another. Thus, the apparatus 100 for monitoring the physical activity may accurately determine whether or not a user actually does exercises, and spoofing of fitness data may be prevented.

The AI learning model may determine the physical activity of the user by inputting the motion sensor data into the first AI learning model and may verify the determined physical activity by inputting the biomedical sensor data into the second AI learning model. The AI learning model may verify the determined physical activity based on the biomedical sensor data, only when the physical activity of the user, which is determined based on the motion sensor data, is included in pre-defined physical activities. For example, when the physical activity of the user determined based on the motion sensor data indicates an exercise, the AI learning model may verify whether or not the user actually does exercise based on the biomedical sensor data.

The AI learning model may determine a first physical activity of the user by inputting the motion sensor data into the first AI learning model, may determine a second physical activity of the user by inputting the biomedical sensor data into the second AI learning model, and may determine whether or not the first physical activity and the second physical activity correspond to each other. The first AI learning model may be trained to determine a physical activity, by using, as training data, the motion sensor data, which is collected from the motion sensor when a person wearing the motion sensor performs various physical activities. The second AI learning model may correspond to a model that is trained to determine a physical activity, by using, as training data, the biomedical sensor data, which is collected from the biomedical sensor when a person wearing the biomedical sensor performs various physical activities.

The AI learning model may determine the physical activity of the user by inputting the motion sensor data and the biomedical sensor data into one AI learning model. Here, the AI learning model may be trained to determine a physical activity, by using, as training data, the motion sensor data and the biomedical sensor data respectively collected from the motion sensor and the biomedical sensor when a person wearing the motion sensor and the biomedical sensor performs various physical activities.

In the embodiment shown in FIG. 4, operations S210 and S220 may be performed at the same time in parallel. Alternatively, operation S220 may be performed after the apparatus 100 determines that the physical activity identified by the first type sensor data does not correspond to one of the pre-defined physical activities, in operation S410, to save the computing power and battery life of the apparatus 100. The processing time of operations S310 and S410 is very short (e.g., 0.1 second) and therefore it may be reasonable to assume that the user engages in the same physical activity while the apparatus 100 performs operations S210 to S410.

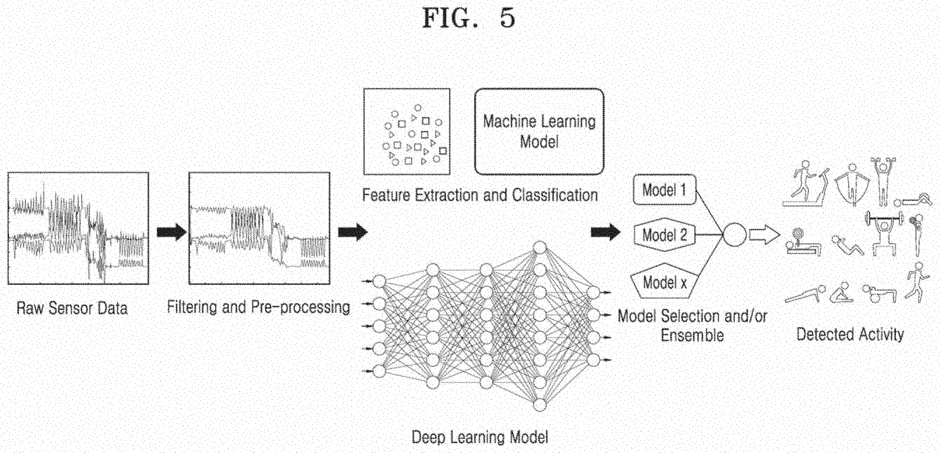

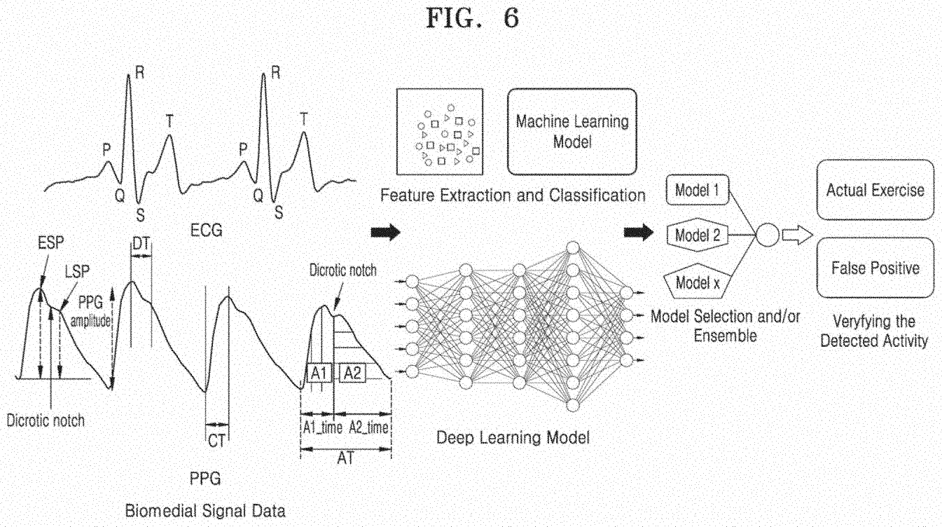

FIG. 5 is a schematic view of a method, performed by the apparatus 100 for monitoring the physical activity, according to an embodiment of the disclosure, of determining a physical activity of a user based on motion sensor data. FIG. 6 is a schematic view of a method, performed by the apparatus 100 for monitoring the physical activity, according to an embodiment of the disclosure, of verifying the physical activity of the user, which is determined based on the motion sensor data, based on biomedical sensor data.

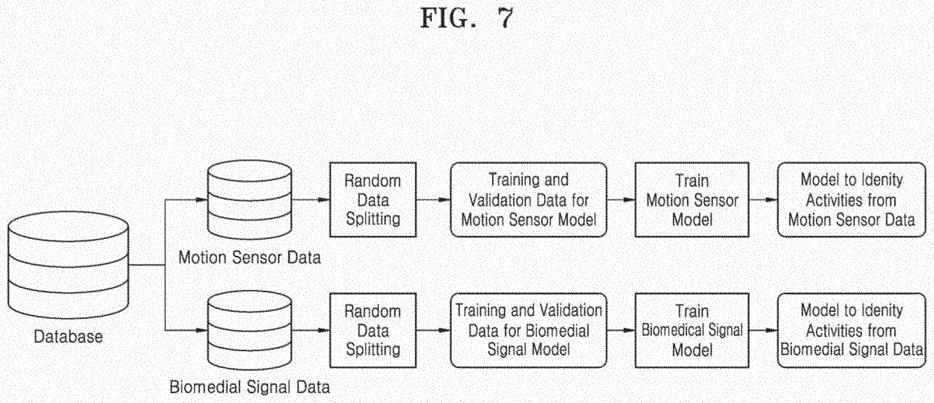

FIG. 7 is a schematic view of a method of training the AI learning model by using the motion sensor data and the biomedical sensor data as training data, according to an embodiment of the disclosure. Referring to FIG. 7, the AI learning model may include two types of AI learning models that are respectively trained based on the motion sensor data and the biomedical sensor data, namely, the first AI learning model and the second AI learning model. In FIG. 7, the first AI learning model and the second AI learning model are referred to as a motion sensor model and a biomedical signal model, respectively. In a training operation, after sufficiently obtaining the motion sensor data and the biomedical sensor data, the motion sensor data and the biomedical sensor data may be arbitrarily divided into a training data set and a verification data set, and after training the AI learning model by using the training data set, the performance of the trained AI learning model may be verified by using the verification data set.

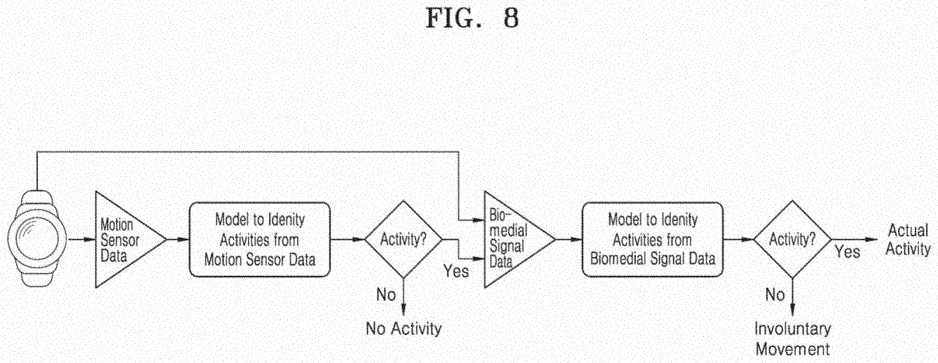

FIG. 8 is a schematic view of a method, performed by the apparatus 100 for monitoring the physical activity, according to an embodiment of the disclosure, of determining a physical activity of a user based on the motion sensor data and the biomedical sensor data. Referring to FIG. 8, the apparatus 100 may verify the determined physical activity based on the biomedical sensor data and the second AI learning model, only when the physical activity of the user, which is determined based on the motion sensor data and the first AI learning model, is included in pre-defined physical activities.

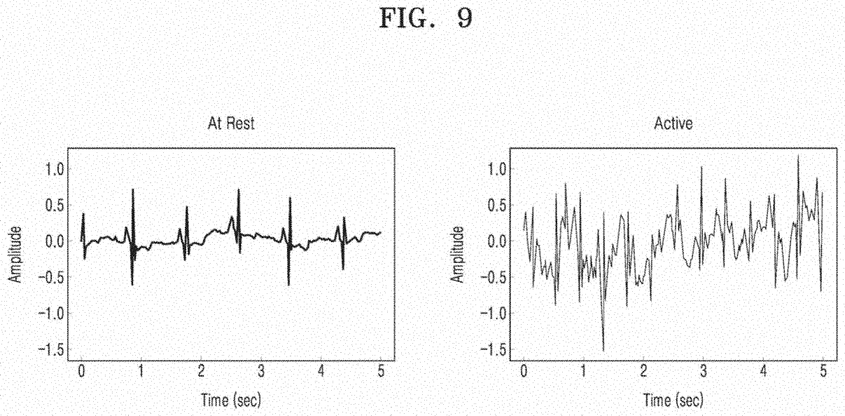

FIG. 9 shows ECG signals when a user takes a rest and when the user does exercise. Referring to FIG. 9, the ECG signals when the user takes a rest and when the user does exercise are greatly different from each other, and thus, the apparatus 100 for monitoring the physical activity may verify whether or not the user does exercise, based on ECG sensor data.

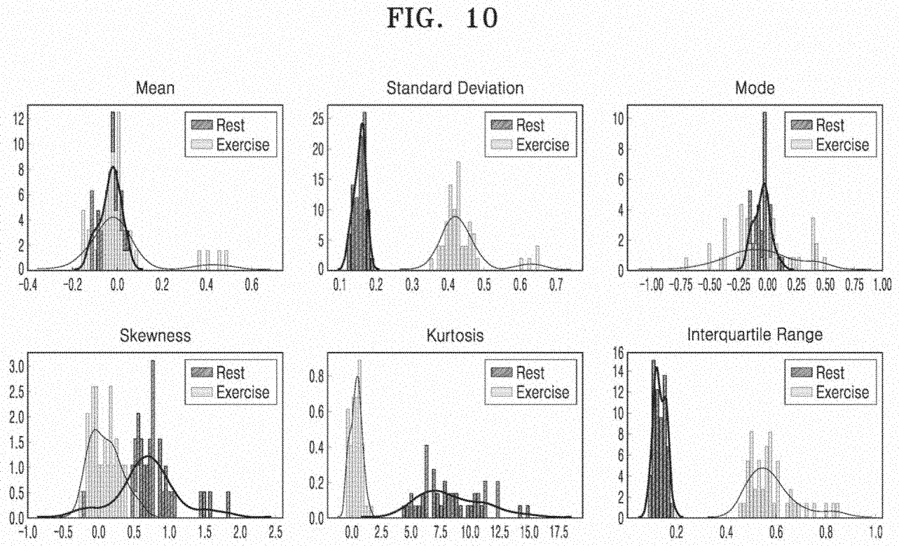

FIG. 10 shows statistical characteristics of ECG signals when a user takes a rest and when the user does exercise. Referring to FIG. 10, the mean, the mode, and the skewness from among the statistical characteristics of the ECG signals may greatly overlap between when the user takes a rest and when the user does exercise. However, the standard deviation, the kurtosis, and the interquartile range may be greatly different between the two cases. Thus, the apparatus 100 for monitoring the physical activity may relatively more accurately verify whether or not the user does exercise by using at least one of the standard deviation, the kurtosis, or the interquartile range of the ECG sensor data.

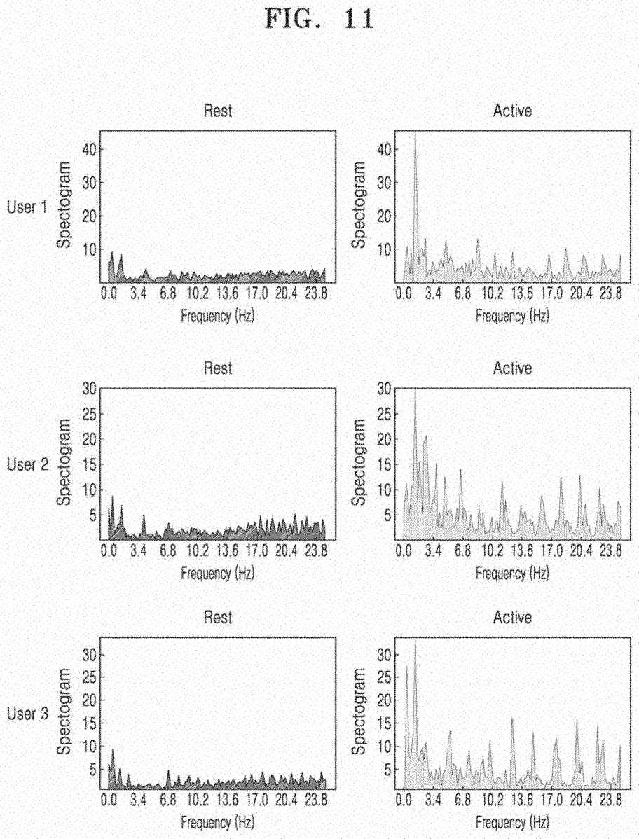

FIG. 11 shows frequency spectrums of ECG signals when three users take a rest and when the users does exercise. The frequency spectrums of the ECG signals illustrated in FIG. 11 are obtained via a Fourier transform. Referring to FIG. 11, the frequency spectrums of the ECG signals when the user takes a rest and when the user does exercise are greatly different from each other. Thus, the apparatus 100 for monitoring the physical activity may verify whether or not the user does exercise by using frequency domain characteristics of the ECG sensor data.

In addition, the apparatus 100 for monitoring the physical activity may verify the physical activity of the user by using biomedical characteristics of the ECG signals, for example, the QRS-complex peak, the P-wave shape, the distance between R peaks, etc. Also, potential characteristics obtained via deep learning may also be used. For example, an activation value of a hidden node in a deep neural network may be used to analyze the ECG signals. Similarly, an autoencoder structure may be used.

These aspects may be likewise applied to PPG signals. Statistical characteristics and frequency domain characteristics of the PPG signals, or potential characteristics obtained from a deep learning model may be used. Also, biomedical characteristics of the PPG signals, such as the diastolic peak, the systolic peak, the diastolic notch, or the distance therebetween, may be used.

Algorithms, such as logistic regression and its transforms, a tree-based algorithm and its transforms, or a gradient boosting method, may be used in machine learning. Also, a deep learning model including a DNN model or an RNN model may be used. These models may be ensembled via bagging or boosting. Continual learning may be used to adapt to a change between users or to a change within a user.

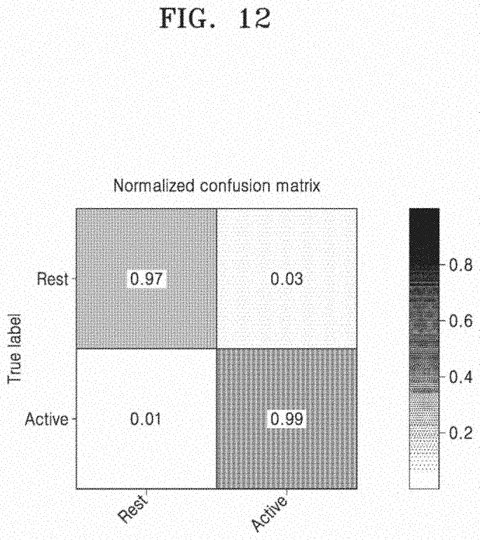

FIG. 12 is a view of a confusion matrix showing performance of the apparatus 100 for monitoring the physical activity using the motion sensor data and the biomedical sensor data together. The AI learning model of the apparatus 100 for monitoring the physical activity of FIG. 12 may correspond to a simple model based on a decision tree, which uses only statistical characteristics of the biomedical sensor data. Referring to FIG. 12, when the motion sensor data and the biomedical sensor data are used together, the accuracy may become high even when the simple model is used.

The apparatus 100 for monitoring the physical activity may not only accurately determine a physical activity of a user by using the motion sensor data and the biomedical sensor data together, but also may accurately provide the user with information related to the physical activity, for example, advice or a recommendation about an exercise that the user is currently performing. For example, when the apparatus 100 for monitoring the physical activity determines that the user actually performs a specific exercise, based on the motion sensor data and the biomedical sensor data, the apparatus 100 for monitoring the physical activity may determine an intensity of the exercise based on a value of the biomedical sensor data. The apparatus 100 for monitoring the physical activity may advise the user to lower the intensity, when the intensity of the exercise is too high, or instruct the user to adjust the intensity of the exercise according to a specific program.

Also, by using these various types of sensor data, a complete profile about the physical activity of the user may be obtained, and accordingly, a more adequate and effective AI learning model may be developed.

As described above, the first type wearable sensor and the second type wearable sensor may include sensors worn on different body parts. For example, the first type wearable sensor may include a motion sensor of a smart watch worn on a wrist, and the second type wearable sensor may include a motion sensor of a smartphone put in a pocket of trousers. Hereinafter, the motion sensors worn on different body parts will be mainly described. However, sensors worn on different body parts may also include sensors for sensing different types of physical quantities.

The apparatus 100 for monitoring the physical activity may receive motion sensor data of a first body part from the motion sensor worn on a first body part of the user, receive motion sensor data of a second body part from the motion sensor worn on a second body part of the user, and input the motion sensor data of the first body part and the motion sensor data of the second body part into the AI learning model to determine the physical activity of the user. Here, the first body part and the second body part may indicate different body parts. In this case, because the physical activity of the user may be determined by using the motion sensor data of the plurality of body parts, compared to a case where only motion sensor data of one body part is used, the physical activity may be more accurately determined and more diverse and complex physical activities may be determined.

The AI learning model may determine the physical activity of the user by inputting the motion sensor data of the first body part and the motion sensor data of the second body part into one AI learning model. Here, the AI learning model may correspond to a model that is trained to determine a physical activity, wherein the AI learning model may be trained by using, as training data, the motion sensor data collected from the motion sensors worn on the first and second body parts of a person when the person wearing the motion sensors on the first and second body parts perform various physical activities. However, motion sensors worn to three or more than three body parts may also be used.

In particular, the whole body physical activity of a user may be relatively more accurately determined by using motion sensor data of a plurality of body parts. The whole body physical activity may indicate an activity in which a plurality of body parts are engaged. Here, the plurality of body parts being engaged in the activity may not necessarily denote that all of the corresponding body parts are in motion. Rather, it may denote that the activity is defined by all of the corresponding body parts engaged therein. For example, the whole body physical activity may indicate a physical activity in which arms move in a predetermined pattern and legs do not move, as in the case of a bench-press exercise. When data of a motion sensor worn on an arm and data of a motion sensor worn on a leg are used, a push-up exercise in which arms move similarly to the bench-press exercise, but legs also move, may be distinguished from the bench-press exercise. The whole body physical activity may include a physical activity, in which no body parts move, like sleeping.

According to an embodiment of the disclosure, the whole body physical activity may indicate an activity in which at least a portion of the upper body and at least a portion of the lower body are engaged. The whole body physical activity may indicate an activity in which a torso is engaged. The whole body physical activity may indicate an activity in which most body parts are engaged. The whole body physical activity may indicate an activity in which all body parts are engaged. The whole body physical activity may include running, an elliptical trainer exercise, football, swimming, etc.

The AI learning model may determine the whole body physical activity of the user by inputting motion sensor data of different body parts into one AI learning model. That is, the AI learning model may include a first AI learning model and may determine the whole body physical activity of the user by inputting the motion sensor data of the first body part and the motion sensor data of the second body part into the first AI learning model. Here, the whole body physical activity may indicate an activity in which the first body part and the second body part are engaged.

As the number of sensors worn on different body parts of a user is increased, the physical activity may be more accurately determined. However, because users normally do not use many wearable devices, it may be required to accurately determine a physical activity as possible by using a less number of wearable devices. Thus, the apparatus 100 for monitoring the physical activity may use a motion sensor of a smartphone, which is a device most frequently used by users, as a wearable sensor. That is, the first type wearable sensor may include a smartphone motion sensor included in the smartphone, the first type sensor data may include smartphone motion sensor data received from the smartphone motion sensor of the smartphone, and the first AI learning model of the AI learning model may determine the physical activity of the user based on the smartphone motion sensor data.

Unlike other general wearable devices worn on a wrist, such as a smart watch or a smart band, the smartphone may be kept in different body parts according to a user or a situation. Thus, the apparatus 100 for monitoring the physical activity may have to know in which location the user keeps the smartphone. To this end, the apparatus 100 for monitoring the physical activity may receive, from the user, a location in which the user keeps the smartphone, and according to an embodiment of the disclosure, may instruct the user to keep the smartphone in a specific location (for example, a trouser pocket) of the body.

The AI learning model may estimate the location in which the smartphone is kept, based on various sensor data received from wearable devices worn on the user, the wearable devices including the smartphone. For example, the AI learning model may determine a body part on which the smartphone is worn, based on the smartphone motion sensor data received from the smartphone motion sensor. The AI learning model may determine the body part on which the smartphone is worn, based on the smartphone motion sensor data received from the smartphone motion sensor and the second type motion sensor data. In this case, the AI learning model may include an AI learning model trained to determine the body part on which the sensor is worn, wherein the AI learning model is trained by using, as training data, sensor data, which is collected from sensors worn on different body parts of a person when the person performs various physical activities.

The apparatus 100 for monitoring the physical activity may obtain information about an activity of the upper body of the user from a smart watch frequently used by users, and thus, may use the motion sensor data of the smartphone worn on the lower body of the user to obtain information about an activity of the lower body of the user. That is, the first AI learning model may determine a motion of the lower body of the user based on the smartphone motion sensor data received from the smartphone motion sensor. When the apparatus 100 for monitoring the physical activity receives an input that the user wears the smartphone on the lower body of the user, or when the apparatus 100 for monitoring the physical activity determines that the user wears the smartphone on the lower body of the user based on the smartphone motion sensor data, the first AI learning model may determine the motion of the lower body of the user based on the smartphone motion sensor data. The first AI learning model may determine a motion of legs of the user based on the smartphone motion sensor data.

The apparatus 100 for monitoring the physical activity may instruct the user to wear the smartphone on the torso (for example, a shirt pocket) in order to obtain information about an activity of the torso of the user. However, in order to accurately determine various physical activities of the user, it is more advantageous to obtain information about the activity of the lower body (or legs) than to obtain information about the activity of the torso. Thus, the apparatus 100 for monitoring the physical activity may instruct the user to wear the smartphone on the lower body of the user and may estimate the activity of the torso or the whole body of the user based on the smartphone worn on the lower body of the user. In particular, when information about an activity of the upper body of the user is obtained via the smart watch, etc., the activity of the torso or the whole body of the user may be estimated from the information about the activity of the upper body and the information about the activity of the lower body, the information about the activity of the lower body being obtained from the smartphone. That is, the first body part and the second body part may not include the torso, and the first AI learning model may determine a motion of the torso or the whole body of the user, based on the motion sensor data of the first body part and the motion sensor data of the second body part. One of the first body part and the second body part may be included in the upper body and the other may be included in the lower body.

People tend to wear a smart watch on a hand that they do not frequently use. For example, a right-handed person may normally wear the smart watch on the left wrist. On the contrary, people tend to put a smartphone in a trouser pocket at a side of a hand that they frequently use. For example, a right-handed person may normally keep the smartphone in a right trouser pocket. In other words, the smart watch may be worn on one side on the upper body, and the smartphone may be worn on the other side on the lower body. Here, “one side” refers to either one of a left side of the body or a right side of the body, and “the other side” refers to a side opposite to the side. As described above, when different sensors are worn on different sides, namely, the left side and the right side, the motion of the torso or the whole body may be easily estimated from data of these sensors. Also, when different sensors are worn on different body parts, the motion of the torso or the whole body may be easily estimated from sensor data. Particularly, when different sensors are worn on different body parts between the upper body and the lower body, the motion of the torso or the whole body may be relatively more easily estimated from the sensor data.

Thus, the first type wearable sensor may include a first side motion sensor of the first body part located at a first side of the first body part, and the second type wearable sensor may include a second side motion sensor of the second body part located at a second side of the second body part, wherein the first side and the second side are opposite to each other. Also, the first AI learning model may determine the motion of the torso or the whole body of the user, based on first side motion sensor data of the first body part received from the first side motion sensor of the first body part and second side motion sensor data of the second body part received from the second side motion sensor of the second body part. Here, the first body part and the second body part indicate different body parts not including the torso (for example, arms and legs). One of the first body part and the second body part may be included in the upper body and the other may be included in the lower body.

Generally, people possess one smart watch and one smartphone. Thus, the apparatus 100 for monitoring the physical activity may obtain information about an activity of an arm by using the smart watch and information about an activity of a leg by using the smartphone. In this case, it may be difficult to determine activities of the other arm and the other leg, or an activity of the torso, and thus, it may be difficult to accurately determine the physical activity of the user.

In order to accurately determine the physical activity of the user, the apparatus 100 for monitoring the physical activity may use a sensor of an earphone, which is another device that is frequently kept by users. That is, the second type wearable sensor may include an earphone motion sensor included in the earphone, and the second type sensor data may include earphone motion sensor data received from the earphone motion sensor. The earphone may include an audio device worn on an ear and may include a headphone, earbuds, a canalphone, a headset, or a bone conduction headphone. The AI learning model may determine the physical activity of the user based on the earphone motion sensor data received from the earphone motion sensor.

An earphone is worn on the head of the user, and thus, the AI learning model may determine a motion of the head of the user based on the earphone motion sensor data received from the earphone motion sensor. The head of the user, on which the earphone is worn, is on the central axis of the torso of the user, and thus, the AI learning model may determine a motion of the torso or the whole body of the user based on the earphone motion sensor data. The AI learning model may determine a direction of the body of the user or vertical symmetry of a physical activity based on the earphone motion sensor data.

The vertical symmetry of the physical activity may correspond to a binary value indicating symmetry or asymmetry, a numerical value (for example, a value between 0 and 1) indicating a degree of asymmetry, or a vector indicating a degree and a direction of asymmetry. The AI learning model may determine the vertical symmetry of the physical activity based on vertical symmetry of a motion indicated by the earphone motion sensor data. The AI learning model may or may not use an AI learning model to determine the vertical symmetry of the physical activity.

An earphone is often worn on both ears, and thus, based on motion sensor data of a left side earphone and a right side earphone, the motion or the direction of the head, the torso, or the whole body of the user, or the vertical symmetry of the physical activity may be relatively more accurately determined. That is, the earphone motion sensor may include a left side earphone motion sensor included in the left side earphone and a right side earphone motion sensor included in the right side earphone. The earphone motion sensor data may include left side earphone motion sensor data received from the left side earphone motion sensor and right side earphone motion sensor data received from the right side earphone motion sensor. Also, the AI learning model may determine the physical activity of the user based on the left side earphone motion sensor data and the right side earphone motion sensor data.

The AI learning model may determine the vertical symmetry of the physical activity based on a degree of similarity between the left side earphone motion sensor data and the right side earphone motion sensor data, or a degree of similarity between motions indicated by the left side earphone motion sensor data and the right side earphone motion sensor data. For example, when the left side earphone motion sensor data and the right side earphone motion sensor data are similar to each other, the AI learning model may determine that the physical activity is vertically symmetrical, and when the left side earphone motion sensor data and the right side earphone motion sensor data are greatly different from each other, the AI learning model may determine that the physical activity is not vertically symmetrical.

The AI learning model may determine the vertical symmetry of the physical activity based on the degree of similarity between the left side earphone motion sensor data and the right side earphone motion sensor data, and a degree of vertical symmetry of the motion indicated by the earphone motion sensor data. For example, the AI learning model may determine that the physical activity is vertically symmetrical, when the motions indicated by the left side earphone motion sensor data and the right side earphone motion sensor data are vertically symmetrical and are similar to each other.

When a sensor device, such as a smart watch or a smartphone, is worn on only one side of the body, the AI learning model may determine an activity of the other side of the body based on the earphone motion sensor data. That is, the first type wearable sensor may include a first side motion sensor at a first side of the body, and the first AI learning model of the AI learning model may determine a motion of a second side of the body based on first side motion sensor data received from the first side motion sensor and the earphone motion sensor data, wherein the first side and the second side are opposite to each other. Here, the motion of the second side of the body may include a motion of the second side of a body part corresponding to a body part, on which the first side motion sensor is worn.

The AI learning model may verify the physical activity determined based on a motion sensor worn on a side of body parts other than ears, based on the earphone motion sensor data. That is, the first type wearable sensor may include the one-sided motion sensor at one side of the body, and the second type wearable sensor may include the earphone motion sensor included in the earphone. Also, the AI learning model may determine the physical activity of the user by inputting the one-sided motion sensor data received from the one-sided motion sensor into the first AI learning model and may verify the determined physical activity based on the earphone motion sensor data received from the earphone motion sensor. Here, the one-sided motion sensor may include a plurality of motion sensors. For example, the AI learning model may verify, based on the earphone motion sensor data, the physical activity determined based on motion sensor data of a smart watch worn on a wrist and a smart phone worn on a leg of the other side. Only when the physical activity of the user, which is determined based on the one-sided motion sensor data, is included in pre-defined physical activities, the AI learning model may verify the determined physical activity based on the earphone motion sensor data. The AI learning model may determine a first physical activity of the user by inputting the one-sided motion sensor data into the first AI learning model, determine a second physical activity of the user by inputting the earphone motion sensor into the second AI learning model, and may determine whether or not the first physical activity and the second physical activity correspond to each other.

Because many exercises are vertically symmetric, the physical activities may be verified based on vertical symmetry. Further, exercises which are vertically asymmetric may also be verified based on the vertical symmetry. That is, the AI learning model may verify the physical activity determined based on the one-sided motion sensor, based on vertical symmetry of the physical activity, the vertical symmetry of the physical activity being determined based on the earphone motion sensor data. Here, the AI learning model may take into account the vertical symmetrical characteristics of the determined physical activity.

According to an embodiment of the disclosure, the AI learning model may determine whether or not the vertical symmetry of the physical activity determined based on the one-sided motion sensor corresponds to the vertical symmetry of the physical activity, which is determined based on the earphone motion sensor data. For example, in a case where the physical activity determined based on the motion sensor data received from a smart watch and a smartphone is a dumbbell curl exercise, when the physical activity is determined to be vertically symmetric based on the earphone motion sensor data, it may be determined that the dumbbell curl exercise is rightly determined.

According to an embodiment of the disclosure, the AI learning model may determine whether or not a vertical symmetrical value or vector of the physical activity determined based on the one-sided motion sensor corresponds to the vertical symmetry of the physical activity, which is determined based on the earphone motion sensor data. For example, in a case where the physical activity determined based on the motion sensor data received from the smart watch and the smartphone is an elliptical exercise, when a vertical symmetrical value of the physical activity determined based on the earphone motion sensor data is near 0 or excessively great, it may be determined that the elliptical exercise is wrongly determined, and when the vertical symmetrical value of the physical activity determined based on the earphone motion sensor corresponds to a general numerical value of the elliptical exercise, it may be determined that the elliptical exercise is rightly determined.

The AI learning model may determine the physical activity of the user by inputting the one-sided motion sensor data received from the one-sided motion sensor together with the vertical symmetry of the physical activity determined based on the earphone motion sensor data into the AI learning model. That is, the AI learning model may include the first AI learning model and may determine the physical activity of the user by inputting the vertical symmetry of the physical activity determined based on the earphone motion sensor data and the one-sided motion sensor data into the first AI learning model.

The AI learning model may determine the physical activity of the user by inputting the one-sided motion sensor data received from the one-sided motion sensor together with the earphone motion sensor data received from the earphone motion sensor into the AI learning model. That is, the AI learning model may include the first AI learning model and may determine the physical activity of the user by inputting the one-sided motion sensor data and the earphone motion sensor data into the first AI learning model.

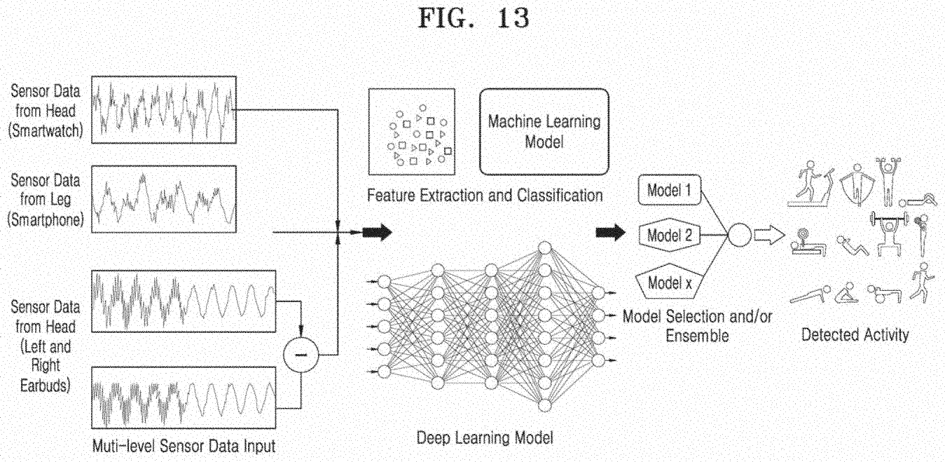

FIG. 13 is a schematic view of a method, performed by the apparatus 100 for monitoring the physical activity, according to an embodiment of the disclosure, of determining a physical activity of a user based on data of sensors worn on a plurality of body parts. Referring to FIG. 13, the apparatus 100 for monitoring the physical activity may determine the physical activity of the user based on sensor data received from a smart watch worn on a wrist, a smartphone worn on a leg, and right and left earbuds worn on the head.

Various embodiments may be realized by combining various configurations described above. For example, the AI learning model may determine the physical activity of the user based on motion sensor data received from the smart watch worn on a left wrist, EMG sensor data, and motion sensor data received from the smartphone kept in a right trouser pocket, and then, may verify the determined physical activity based on vertical symmetry determined based on motion sensor data received from the earphone. When the verified physical activity is an aerobic exercise, the verified physical activity may be further verified based on PPG sensor data received from the smart watch, and the verified physical activity may be further again verified based on ECG sensor data received from the smart watch and EOG sensor data received from smart glasses. As another example, the AI learning model may determine the physical activity of the user by inputting all of the motion sensor data received from the smart watch, the smartphone, and the earphone, the PPG sensor data and the ECG sensor data received from the smart watch, and the EOG sensor data received from the smart glasses into one AI learning model. The AI learning model may include a single AI learning model or a plurality of AI learning models and may select and use an appropriate AI learning model based on situations.

Types of sensor data which may be obtained by the apparatus 100 for monitoring the physical activity may vary according to situations, because different sensors may be included in various wearable devices, users may have different wearable devices, the same user may wear different devices depending on occasions, and one or more of the devices or sensors worn by the user may be broken or may run out of batteries so as not to normally operate. However, when different AI learning models are used, depending on the types of obtained sensor data, a great number of AI learning models may have to be required. Thus, while the apparatus 100 for monitoring the physical activity may use the AI learning model receiving various types of sensor data, the apparatus 100 for monitoring the physical activity may directly generate and input types of sensor data that are not obtained into the AI learning model. That is, the apparatus 100 may determine the physical activity of the user by inputting the first type sensor data received from the first type wearable sensor and the second type sensor data received from the second type wearable sensor into the AI learning model portion. At the same time, when the second type sensor data is not normally or currently received because the second type sensor malfunctions or does not operate in a preset manner, the apparatus 100 may generate the second type sensor data based on a previously obtained first type sensor data or a previously obtained second sensor data, and input the generated second type sensor data into the AI learning model portion.

According to an embodiment of the disclosure, the second type sensor data may be generated as a default value. According to an embodiment of the disclosure, the second type sensor data may be generated as a value estimated based on a value of the second type sensor data previously received. According to an embodiment of the disclosure, the second type sensor data may be generated as a value estimated based on the first type sensor data received from the first type wearable sensor. Here, the first type sensor data may include a plurality of types of sensor data. According to an embodiment of the disclosure, the second type sensor data may be generated as a value estimated based on the value of the second type sensor data previously received and the first type sensor data received from the first type wearable sensor.

Embodiments of the disclosure may be realized as a computer-executable code recorded on a computer-readable recording medium. The computer-readable recording medium may include a magnetic medium, an optical medium, a read-only memory (ROM), a random-access memory (RAM), etc. The computer-readable recording medium may include the form of a non-transitory recording medium. Here, the expression of “non-transitory recording medium” may only indicate that the medium is a tangible device, rather than a signal (for example, an electromagnetic wave), and does not distinguish a semi-permanent storage of data in the recording medium and a temporary storage of data in the recording medium. For example, the “non-transitory recording medium” may include a buffer in which data is temporarily stored.

According to an embodiment of the disclosure, methods according to various embodiments of the disclosure may be provided as a computer program product. The computer program product may be purchased as a product between a seller and a purchaser. The computer program product may be distributed by being stored in a computer-readable recording medium, may be distributed through an application store (e.g. a Play Store™), or may be directly or through online distributed between two user devices (for example, smartphones). In the case of online distribution, at least a portion of the computer program product (for example, a downloadable application) may be at least temporarily stored in the computer-readable recording medium, such as a server of a manufacturer, a server of the application store, or a memory of a broadcasting server, or may be temporarily generated.

According to an embodiment of the disclosure, a method and an apparatus for monitoring a physical activity based on a wearable sensor are provided to relatively more accurately monitor the physical activity of a user.

While not restricted thereto, embodiments can be implemented as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, embodiments may be written as a computer program transmitted over a computer-readable transmission medium, such as a carrier wave, and received and implemented in general-use or special-purpose digital computers that execute the programs. Moreover, it is understood that in example embodiments, one or more units of the above-described apparatuses and devices can include circuitry, a processor, a microprocessor, etc., and may execute a computer program stored in a computer-readable medium.

The foregoing embodiments are merely examples and are not to be construed as limiting. The present teaching can be readily applied to other types of apparatuses. Also, the description of the embodiments is intended to be illustrative, and not to limit the scope of the claims, and many alternatives, modifications, and variations will be apparent to those skilled in the art.

What is claimed is:

1. An apparatus for monitoring a physical activity, the apparatus comprising:
a memory storing one or more instructions; and
at least one processor configured to execute the one or more instructions stored in the memory, to:
obtain first type sensor data from a first type wearable sensor;
obtain second type sensor data from a second type wearable sensor; and
identify the physical activity of a user by using at least one artificial intelligence learning model, the first type sensor data, and the second type sensor data.

2. The apparatus of claim 1, wherein the at least one artificial intelligence learning model includes a first artificial intelligence learning model and a second artificial intelligence learning model, and
the at least one processor is further configured to execute the one or more instructions to:
identify the physical activity of the user by inputting the first type sensor data into the first artificial intelligence learning model; and
verify the identified physical activity by inputting the second type sensor data into the second artificial intelligence learning model.

3. The apparatus of claim 2, wherein the at least one processor is further configured to execute the one or more instructions to verify the identified physical activity by inputting the second type sensor data into the second artificial intelligence learning model, when the physical activity of the user identified based on the first type sensor data is one of a plurality of pre-defined physical activities.

4. The apparatus of claim 1, wherein the at least one artificial intelligence learning model includes a first artificial intelligence learning model and a second artificial intelligence learning model, and
the at least one processor is further configured to execute the one or more instructions to:
identify a first physical activity of the user by inputting the first type sensor data into the first artificial intelligence learning model;
identify a second physical activity of the user by inputting the second type sensor data into the second artificial intelligence learning model; and
determine whether the first physical activity corresponds to the second physical activity correspond.

5. The apparatus of claim 1, wherein the at least one artificial intelligence learning model includes a first artificial intelligence learning model, and
the at least one processor is further configured to execute the one or more instructions to identify the physical activity of the user by inputting the first type sensor data and the second type sensor data into the first artificial intelligence learning model.

6. The apparatus of claim 5, wherein, when a current value of the second type sensor data is not obtained, the at least one processor is further configured to execute the one or more instructions to identify the physical activity of the user by inputting, into the at least one artificial intelligence learning model, an estimated value of the second type sensor data, the estimated value being estimated based on at least one of a previous value of the second type sensor data or the first type sensor data.

7. The apparatus of claim 1, wherein the first type wearable sensor includes a motion sensor, and the first type sensor data includes motion sensor data obtained from the motion sensor, and
the second type wearable sensor includes a biomedical sensor, and the second type sensor data includes biomedical sensor data obtained from the biomedical sensor.

8. The apparatus of claim 1, wherein the first type wearable sensor includes a first motion sensor worn on a first body part of the user, and the first type sensor data includes first body part motion sensor data obtained from the first motion sensor worn on the first body part of the user, and
the second type wearable sensor includes a second motion sensor worn on a second body part of the user, and the second type sensor data includes second body part motion sensor data obtained from the second motion sensor worn on the second body part of the user.

9. The apparatus of claim 8, wherein the at least one artificial intelligence learning model includes a first artificial intelligence learning model, and
the at least one processor is further configured to execute the one or more instructions to identify a whole body physical activity of the user by inputting the first body part motion sensor data and the second body part motion sensor data into the first artificial intelligence learning model.

10. The apparatus of claim 8, wherein the first type wearable sensor includes a smartphone motion sensor included in a smartphone, and the first type sensor data includes smartphone motion sensor data obtained from the smartphone motion sensor,
the at least one artificial intelligence learning model includes a first artificial intelligence learning model, and
the at least one processor is further configured to execute the one or more instructions to identify a body part, on which the smartphone is worn, based on the smartphone motion sensor data, by using the first artificial intelligence learning model.

11. The apparatus of claim 8, wherein the first body part and the second body part do not include a torso,
the at least one artificial intelligence learning model includes a first artificial intelligence learning model, and
the at least one processor is further configured to execute the one or more instructions to identify a motion of the torso of the user based on the first body part motion sensor data and the second body part motion sensor data, by using the first artificial intelligence learning model.

12. The apparatus of claim 11, wherein the first type wearable sensor includes a first side motion sensor of the first body part at a first side of the first body part, and the first type sensor data includes first side motion sensor data of the first body part obtained from the first side motion sensor of the first body part,
the second type wearable sensor includes a second side motion sensor of the second body part at a second side of the second body part, and the second type sensor data includes second side motion sensor data of the second body part obtained from the second side motion sensor of the second body part, wherein the second side is opposite to the first side, and
the at least one processor is further configured to execute the one or more instructions to identify the motion of the torso of the user based on the first side motion sensor data of the first body part and the second side motion sensor data of the second body part, by using the first artificial intelligence learning model.

13. The apparatus of claim 8, wherein the second type wearable sensor includes an earphone motion sensor included in an earphone, and the second type sensor data includes earphone motion sensor data obtained from the earphone motion sensor.

14. The apparatus of claim 13, wherein the earphone motion sensor includes a left earphone motion sensor included in a left earphone portion and a right earphone motion sensor included in a right earphone portion, and
the earphone motion sensor data includes left earphone motion sensor data obtained from the left earphone motion sensor and right earphone motion sensor data obtained from the right earphone motion sensor.

15. The apparatus of claim 13, wherein the first type wearable sensor includes a first side motion sensor at a first side of the first body part, and the first type sensor data includes first side motion sensor data obtained from the first side motion sensor,
the at least one artificial intelligence learning model includes a first artificial intelligence learning model, and
the at least one processor is further configured to execute the one or more instructions to identify a motion of a second side of the first body part based on the first side motion sensor data and the earphone motion sensor data, by using the first artificial intelligence learning model, wherein the second side is opposite to the first side.

16. The apparatus of claim 13, wherein the at least one processor is further configured to execute the one or more instructions to determine vertical symmetry of the physical activity of the user based on the earphone motion sensor data.

17. The apparatus of claim 16, wherein the first type wearable sensor includes a one-sided motion sensor at a side of the first body part, and the first type sensor data includes one-sided motion sensor data obtained from the one-sided motion sensor,
the at least one artificial intelligence learning model includes a first artificial intelligence learning model, and
the at least one processor is further configured to execute the one or more instructions to:
identify the physical activity of the user by inputting the one-sided motion sensor data into the first artificial intelligence learning model; and
verify the identified physical activity of the user based on the determined vertical symmetry.

18. The apparatus of claim 16, wherein the first type wearable sensor includes a one-sided motion sensor at a side of the second body part, and the first type sensor data includes one-sided motion sensor data obtained from the one-sided motion sensor,
the at least one artificial intelligence learning model includes a first artificial intelligence learning model, and
the at least one processor is further configured to execute the one or more instructions to identify the physical activity of the user by inputting the determined vertical symmetry and the one-sided motion sensor data into the first artificial intelligence learning model.

19. An operating method of an apparatus for monitoring a physical activity, the operating method comprising:
obtaining first type sensor data from a first type wearable sensor;
obtaining second type sensor data from a second type wearable sensor; and
identifying the physical activity of a user by using at least one artificial intelligence learning model, the first type sensor data, and the second type sensor data.

20. The operating method of claim 19, wherein the at least one artificial intelligence learning model includes a first artificial intelligence learning model and a second artificial intelligence learning model, and
the operating method further comprises:
identifying the physical activity of the user by inputting the first type sensor data into the first artificial intelligence learning model; and
verifying the identified physical activity by inputting the second type sensor data into the second artificial intelligence learning model, when the physical activity of the user identified based on the first type sensor data is one of a plurality of pre-defined physical activities.
